(19) United States
(12) Patent Application Publication
(10) Pub. No.: US 2004/0048775 A1
(43) Pub. Date: Mar. 11, 2004

(54) Method of qualifying and maintaining appropriate drug administration

(76) Inventors: Donald M. Casey, Ponte Vedra Beach, FL (US); Jerry B. Hansen, Blue Bell, PA (US); Rosalinda Markels, Lower Gwynedd, PA (US); Cornelius Merlini, Ambler, PA (US)

Correspondence Address: Connolly Bove, Lodge & Hutz, PO Box 2207, Wilmington, DE, 19899-2207, US

(21) Appl. No.: 10/332,253

(22) PCT Filed: Jul. 10, 2001
(86) PCT No.: PCT/US01/21643

(51) Int. Cl. A61K 31/00; G06F 17/60

(52) U.S. Cl. 514/1; 705/2

(57) ABSTRACT

A method (FIG. 1)for reducing consumer errors in selecting a medicine for treatment of asymptomatic or chronic conditions and encouraging appropriate long-term use of the medicine. The first step of the method includes stepwise guidance to understanding label information, which leads the consumer through the criteria for determining whether use of the medicine is appropriate. Another step involves directing the consumer to packaging inserts, including an informational booklet and visual media such as a videotape, further explaining the label information. Yet another step involves communication of incentives to maintain long term usage, including incentives to call a toll-free service to speak to a trained product specialist. For eligible consumers, the product specialist gives advice about proper use of the product. For ineligible consumers, the product specialist advises the consumer to discuss the product and condition with a doctor and facilitates a full purchase price refund.

### BACKGROUND OF THE INVENTION

A. Field of the Invention

The present invention relates generally to criteria for over-the-counter (OTC) administration of medicines, and, more particularly to methods of assuring continued, appropriate use of such medicines.

B. Description of the Related Art

Certain drugs acquire a relative degree of acceptance after years of use. Their efficacy and incidence of side effects makes some of these drugs candidates for administration as over-the-counter medicines.

However, there is still a risk of inappropriate use associated with OTC medicines, particularly those used to treat asymptomatic or chronic conditions such as hypertension, osteoporosis, or cholesterol reduction to prevent heart disease. Unlike dosing for symptomatic, self limited conditions readily recognized by consumers, for example, headaches or heartburn, asymptomatic or chronic conditions pose a real risk of inappropriate administration. Such asymptomatic conditions seldom exhibit symptoms that indicate if, when, or how much medication to take. Hypertension, high cholesterol, and osteoporosis, for exarnple, seldom send signals like headaches or heartburn that guide consumers in drug administration.

With asymptomatic or chronic conditions, consumers need to consider more complex information to determine, first, whether the product is right for them and, second, how to use the product appropriately. Proper guidance to consumers is critical in transitioning a medicine from prescription to over-the-counter use. The United States Food and Drug Administration (FDA) will not allow OTC sale of a medicine for treatment of asymptomatic or chronic conditions unless suitable safeguards are in place to assure the product is right for the consumer and the consumer is properly instructed in its use.

An example of a drug successfully transitioned from prescription to over-the-counter administration is nicotine polacrilex gum. A legal predicate required by the FDA for transitioning this gum to OTC use was the development of consumer education vehicles that provide guidance as to who should use the gum and in what dosage. In 1996, the FDA approved over-the-counter sale of nicotine polacrilex gum sold by SmithKline Beecham under the trademark NICORETTE®. That product contained the traditional dosing and warning labels on the carton, as well as an instructional audiotape and brochure designed to help smokers give up cigarettes. Additional motivational materials mailed over a twelve (12) week period were available by calling a toll free number.

Similarly, a nicotine transdermal system (“nicotine patch”) sold by McNeil Consumer Products Co. under the NICOTROL® mark transitioned from prescription to OTC. It too used an audiotape and instructional materials, coupled with a toll free hot line to answer questions regarding the patch. A personal “quit guide” was also offered to smokers who filled out a questionnaire regarding their smoking habits.

These approaches to transitioning nicotine substitutes from prescription to over-the-counter usage, however, have limited application in treatment of asymptomatic chronic conditions. First, there is no confusion in self-recognizing the desire to quit smoking. Second, the need for a smoking cessation aid is short term. Third, the symptoms of nicotine withdrawal are recognizable as are their disappearance. Asymptomatic or chronic conditions require a set of information to recognize when use of the product is appropriate or not, plus continued motivation and incentives to continuously and correctly administer medicines needed to control such conditions. The present invention addresses those needs.

### SUMMARY OF THE INVENTION

In the treatment of asymptomatic or chronic conditions, consumers must consider relatively complex information to determine first, whether a medicine is right for them and second, how to use the product appropriately. This invention improves that ability to understand complex information by addition of enhancements to the product labeling and by novel materials provided with the product packaging.

These novel materials include a video tape, informational brochure, plus unique materials providing incentives for continued treatment of the asymptomatic or chronic condition. These incentives promote or induce electronic communication by the consumer with a trained product specialist, for example through use of a toll-free telephone number or web site, to gain information regarding initial and continued use of the medicine. The trained product specialist is a person having access to specialized information about the initial and long-term use of the medicine. One difference between the toll free telephone service of the related art and that of this invention is the latter's incentive is used to achieve specific purposes. These purposes are to aid with the consumer decisions on whether the product is appropriate for them and, in a proactive way, to assist with proper long-term use.

One application of this invention is in the treatment of high cholesterol. Consumers are aware that cholesterol is a health risk and many of these individuals are interested in a nonprescription cholesterol-reducing product to maintain healthy cholesterol levels. Lovastatin (10 mg to 80 mg) is a prescription drug, which is an effective cholesterol-lowering agent. It is sold in the United States by Merck & Co., Inc. under the MEVACOR® label. Labeling of lovastatin for potential OTC sale has been developed and tested. When tested, most consumers made an appropriate decision to use/purchase the product. However, a subset of consumers made an inappropriate decision to use the product despite label warnings not to do so, such as having cholesterol levels too high for the nonprescription product or having an exclusionary medical condition such as hepatitis or other liver disease.

Other examples of cholesterol lowering agents include but are not limited to statins in their lactonized or dihydroxy open acid forms and pharmaceutically acceptable salts and esters thereof, such as simvastatin (US tradename ZOCOR®, see U.S Pat. No. 4,444,784); dihydroxy open-acid simvastatin, particularly the ammonium or calcium salts thereof; pravastatin, particularly the sodium salt thereof (US tradename PRAVACHOL®, see U.S. Pat. No. 4,346,227); fluvastatin, particularly the sodium salt thereof (US tradename LESCOL®, see U.S. Pat. No. 5,354,772); atorvastatin, particularly the calcium salt thereof (US tradename LIPITOR®, see U.S. Pat. No. 5,273,995); cerivastatin, particularly the sodium salt thereof (US tradename BAYCHOL®, see U.S. Pat. No. 5,177,080); pitavastatin also referred to as NK-104 (see PCT international publication number WO 97/23200); and ZD-4522 (see U.S. Pat. No. 5,260,440).

This invention describes a method that has been shown to reduce consumer errors in selecting the product and to encourage appropriate long-term use. The first step of the method includes a stepwise guide to understanding label information which leads the consumer through the criteria for determining whether use of this drug is appropriate. The next step involves directing the consumer to package inserts, (consumer information leaflet), including a cholesterol information booklet and videotape, further explaining the label information. Yet another step involves communication of incentives to maintain long term usage, including incentives to encourage communication with a trained product specialist. For eligible consumers, the product specialist gives answers to questions about proper use of the product. For ineligible consumers, the product specialist advises the consumer to discuss the product and the condition with a doctor. The trained product specialist interviews the consumer via telephone, internet or other interactive communication media guided by a scripted series of questions that represent all of the conditions for proper use and a computerized program to determine the consumer's eligibility for the product.

To encourage this contact with a product specialist, the consumer is offered unspecified “high value incentives” in materials accompanying the medicine such as a separate incentive coupon. It has been found that the labeling of such incentives as having a “high value” increases the number of consumers who will make contact with the product specialist. That, in turn, decreases the number of consumers who should not take the medicine. While labeling the incentive as “high value” in the preferred embodiment of this invention, details of the incentive and its exact value are unspecified, i.e., not stated on the packaging. Examples of “high value” incentives that can be used with the invention are a free month's supply of the medicine and payment for an analytical test related to administration of the medicine. For example, a blood test to determine cholesterol level would be offered in conjunction with a cholesterol lowering medicine. Another incentive offered to consumers would be full purchase price refund if, after communication with the trained product specialist, it is determined that the consumer should not take the medicine.

It has been found in clinical studies using the invention that about fifty percent (50%) of the consumers remain on the medicine after eighteen (18) months. This is a significant percentage that compares favorably to those who remain on medication for more serious, symptomatic conditions and who are under the direct care and direction of a health professional. In those situations, the professional is typically the vehicle for encouraging continued long term use of a medicine.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention. In the drawings:

FIG. 1 is a schematic drawing illustrating the methods of this invention;

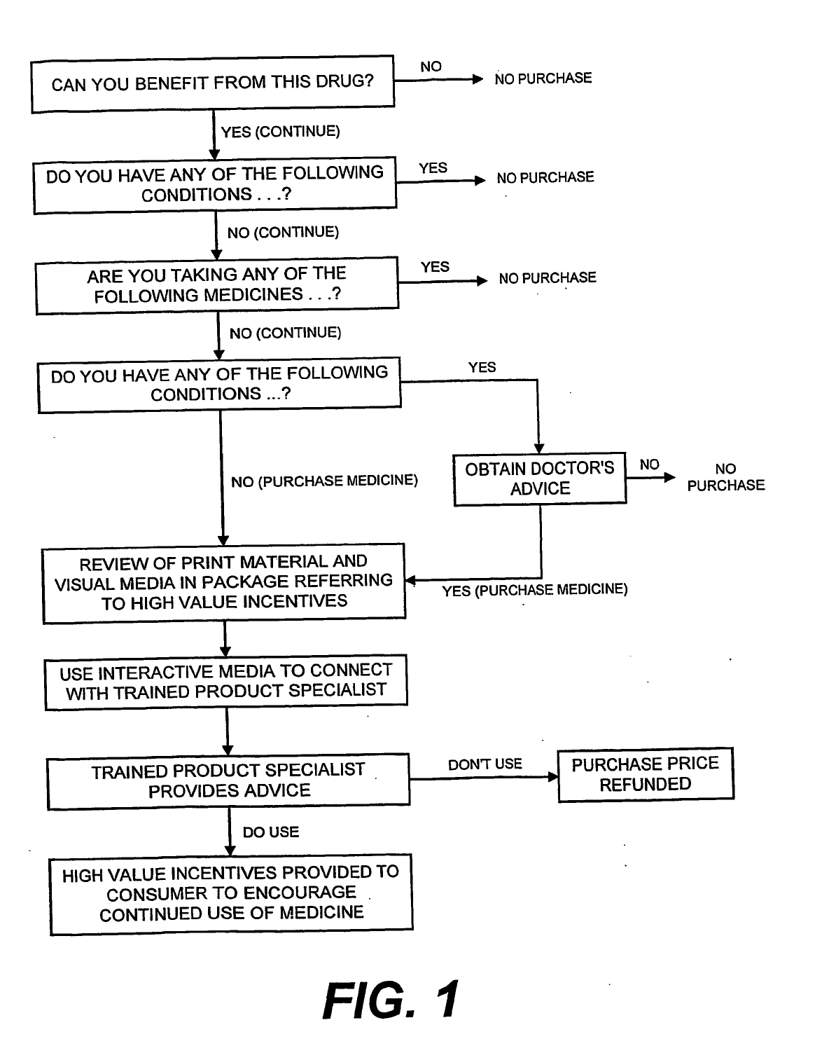

FIG. 2 illustrates the packaging instructions on the carton back label of an OTC medicine to be used pursuant to this invention; and

FIG. 3 illustrates an incentive coupon used to communicate the offer of a “high value” incentive pursuant to this invention.

### DESCRIPTION OF THE PREFERRED EMODIMENTS

Reference will now be made in detail to the present preferred embodiments of the invention, examples of which are illustrated in the accompanying drawings.

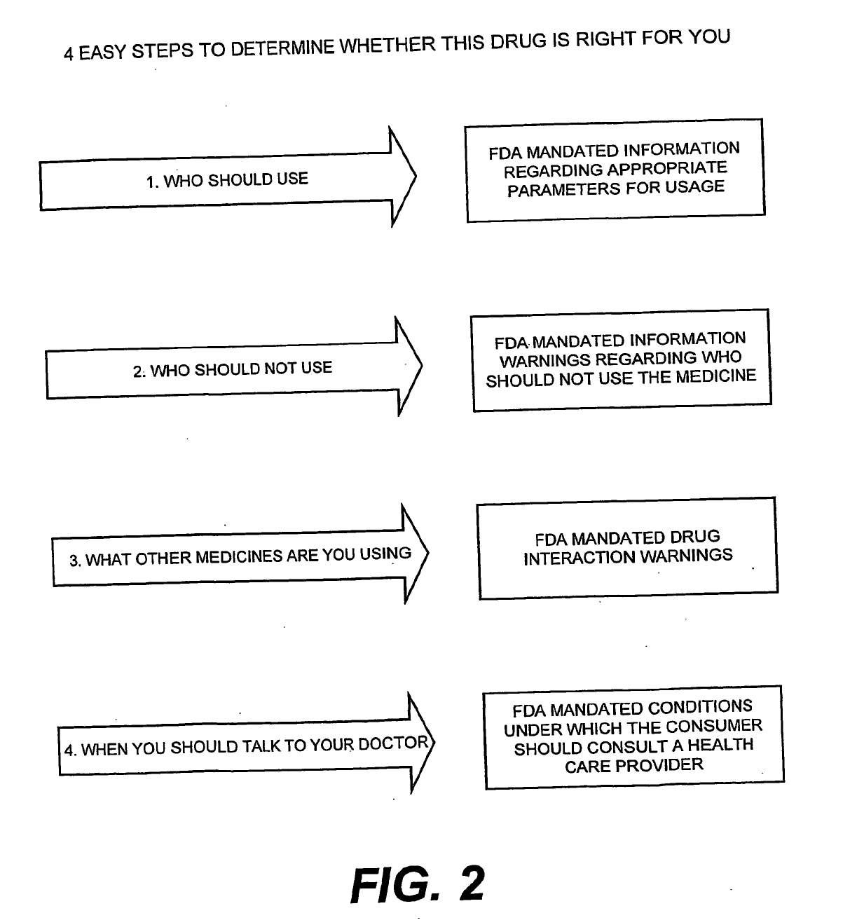

A consumer selecting an over-the-counter medication for treatment of an asymptomatic or chronic condition must receive appropriate information to assist him or her in making an informed selection of such medicines, since this initial selection is typically done without assistance in a drugstore or other retail outlet. The first step of this invention provides clear cut multi-step guidance for making that selection. More particularly, as illustrated in FIG. 2, the back of the carton containing the OTC medicine includes the following four step guidance to the consumer which is complementary to the FDA-mandated Drug Facts format described in 21 C.F.R. §201.66.

Step 1: Criteria for usage. Parameters of the asymptomatic condition, which are treatable with the medicine, are stated, for example, cholesterol levels that warrant treatment. Personal criteria such as age and sex of consumer who can benefit from the medicine can also be stated.

Step 2: Prohibitions on usage. A clear statement of who should not use the OTC medicine is presented in this step of the carton instructions. For example, for a cholesterol reducing medication, the potential user would be warned against use if he or she has hepatitis or liver disease.

Step 3: An interaction guide. Any and all medicines known to have potential adverse reaction with the OTC medication are listed in this step of the carton instruction.

Step 4: Professional consultation criteria. The potential user of the OTC medication is advised in this step to consult a doctor if he or she meets certain medical (e.g. high blood pressure, diabetes, etc.) or societal (more than 3 alcoholic drinks per day) criteria

If the user purchases the OTC medication after reviewing the four step screening on the carton back, he/she is ready to advance to the next step of this invention, more particularly, print and visual media within the carton intended to expand upon and reinforce messages initially presented on the carton back panel. Each carton contains visual media, e.g., a videotape, CD or DVD which visually reinforce the four steps listed on the carton back. The videotape typically portrays actors exchanging information about asymptomatic conditions to be treated by the OTC drug. In the case of a cholesterol-lowering drug, the initial dialog on the tape or other media generally would discuss the circulatory system and the effect of high cholesterol upon that system, that is, eventual artery blockage and heart attacks.

Next, the videotape reinforces the four step selection criteria on the carton back panel by explaining in dialog format which persons should or should not use the OTC medication. It also reinforces any warnings on the carton regarding medical or societal conditions that counsel against use of the medication and/or require consultation with a health care professional before use.

The videotape is supplemented with printed carton inserts, including a brochure that elaborates upon the videotape messages regarding the nature of the condition to be treated, health hints (e.g., diet, exercise, weight control), and helpful dosage and administration advice, for example, visual and other stimuli that can be used to remind a user when to take the medication.

Both the visual media and printed carton inserts include important educational materials that encourage continued proper usage of the medication. This is highly important in the treatment of asymptomatic and chronic conditions where the effect of the OTC medication is not generally perceptible. For example, on a day-to-day basis, the use of an OTC cholesterol reducing medication is not likely to make a person feel any better. That person can only judge the efficacy of the medication by comparing cholesterol levels as determined by periodic blood tests. Aside from such tests, it has been found important to provide consumer motivation to continue treatment when appropriate. Conversely, it is also important to provide motivation to the consumer not to initiate treatment when inappropriate.

Such motivation is provided in this invention. The videotape and printed material within the product carton provide several forms of incentives. First, the consumer is provided with information regarding interactive media such as a toll-free number and/or a web site that will link the consumer with a product specialist who is qualified to counsel the consumer regarding initial and continued use of the OTC medication. The phone number and/or website domain name are preferably selected to designate a particular drug or condition which promotes recognition by the consumer and insures that calls are properly directed to a product specialist fully conversant with the drug. Thus for example, a consumer who dials a telephone number such as 1-800-MEVACOR or types in a domain name such as mevacor.com will be connected with a product specialist familiar with MEVACOR® and criteria for its use. This correlation of interactive media number/name to product specialist either encourages consumer participation in the drug selection process because it insures that a product specialist with full information will be able to answer the consumer's questions on the first call. While the phone number and/or domain name preferably have a sound or connotation corresponding to the medicine in question, any number or name which assures consumer contact with a product specialist familiar with the medicine purchased by the consumer can be used in accordance with this invention.

Unlike typical toll-free calls that merely receive consumer input or complaints, the arrangement contemplated by this invention is truly interactive. The product specialist is trained to give advice about the use of the medication and is available to provide information needed for proper long-term usage of the medication. The videotape and printed material within the carton also alert the consumer to the availability of a partial or full purchase price refund if contact with the product specialist determines that the consumer should not use the medication for any reason. The product specialist can reconfirm the availability of such refund at the outset of or during communication with the customer to further encourage candid discussion of medical conditions with the product specialist. Thus, this invention provides important motivation, i.e., refund, for consumers to communicate with an informed product specialist who can confirm, or, overturn, the consumer's original decision to purchase the medicine.

In the past, the back panel instructions on an OTC product were designed to guide consumers to the proper usage of the product. Consumers who had some difficulty in determining whether a product was right for them had to consult with a health care professional such as a pharmacist or doctor. This interaction, while desirable and necessary in some cases, is not as easy to achieve given the time constraints these professionals are under in today's health care environment.

One advantage of this invention is that it allows individuals who need to confirm their purchase decision to do so in a convenient interactive manner by taking with a trained product specialist via interactive media such as a toll free number or an interactive website. For those consumers who made an incorrect purchase decision, the method allows for a refund of the purchase price. The overall impact of this process is that there will be fewer consumers taking the product incorrectly and more people taking the product correctly, for example, to maintain healthy cholesterol levels.

For those consumers who can benefit from the medication, the product specialist will interview the consumer guided by a scripted series of questions that represent all of the conditions for proper long-term use.

Another incentive offered the consumer is free enrollment in a program designed to encourage continued, informed usage of the medication while providing valuable written materials to the consumer. For example, the program might offer a cookbook on how to prepare low cholesterol meals coupled with a periodic newsletter directed to the health condition being treated by the medication. The newsletter might also contain valuable coupons for a variety of products related to the condition being treated.

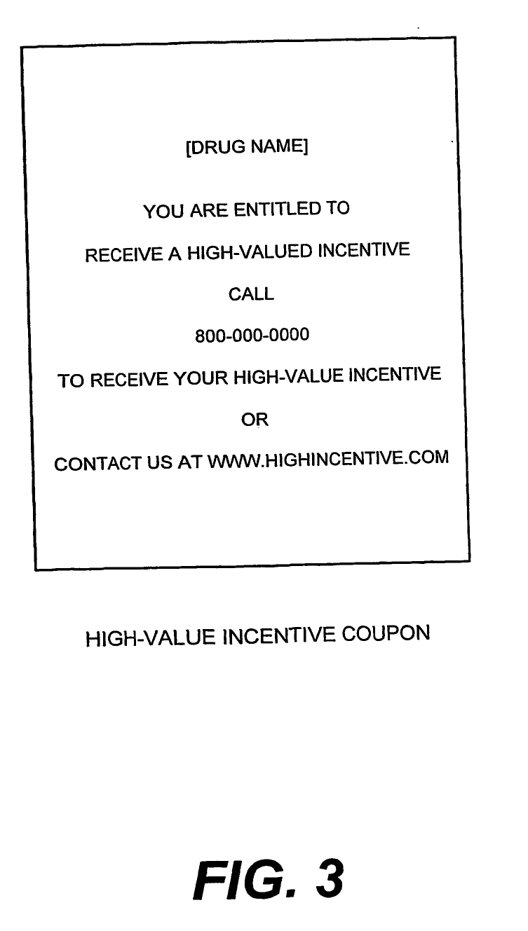

Another incentive offered to the consumer to encourage communication regarding their choice of medication and continued usage of same is the offer of an unspecified “high value incentive” if the consumer calls the toll free number and/or contacts a designated website. This “high value incentive” is not described on, nor in, the materials in the carton containing the medicine. It has been found that such incentives are best communicated in a separate incentive coupon. One example of such a coupon is shown in FIG. 3. The offer of something described as a “high value incentive”, related to the product has been found to prompt a high percentage of consumers into calling a toll free number or contacting a designated website. Such contact and subsequent communication with the product specialist provides a higher level of assurance that the consumer is qualified to take the medication. Such assurance is very important for chronic medications being transitioned from prescription to OTC, and helps to allay fears of regulatory agencies about potential improper use of the medication, for asymptomatic conditions.

The “high value incentives” of this method are typically items having considerable appeal to consumers, while having a manageable cost to the supplier of the medicine. One example of such an incentive would be a free supply of the medication, typically enough for one month's administration. Another example of such an incentive would be discounts on future purchases, of the medicine or other products related to the condition being treated by that medicine.

Another example of a high value incentive is a free blood test or other analytical procedure related to the asymptomatic/chronic condition to be treated with the medication. Such a test provides a strong incentive to continue use of the medication if the test confirms positive results from use of the medication. For example, a blood test that confirms reduced cholesterol levels after use of a cholesterol lowering medication will encourage consumers to continue use of that medication. This is a plus, both for the consumer (better health through lowered cholesterol levels) and the supplier of the medication (continued sales of the medication). A certificate for such a test can be transmitted to the consumer if deemed appropriate by the product specialist.

The invention has been illustrated by detailed description and examples of the preferred embodiment. Various changes in form and detail will be within the skill of persons skilled in the art. Therefore, the invention must be measured by the claims and not by the description of the examples or the preferred embodiments.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of qualifying and promoting appropriate over-the-counter administration of medicines by consumers, comprising the steps of:
a) displaying information on the exterior surfaces of containers for such medicines which information comprises multi-step guidance on qualifying criteria for use of the medicines; and
b) offering incentives to the consumer to induce interactive communication by the consumer with a person having access to specialized information about the initial and long term use of such medicine.

2. The method of claim 1, wherein at least one incentive offered to the consumer comprises an item described as a high value incentive.

3. The method of claim 2, wherein the high value incentive is selected from the group consisting of a free supply of the medicine, discounts on other products related to the condition being treated and analytical tests related to the efficacy of the medicine.

4. The method of claim 1, wherein the multi-step guidance on the container includes criteria for safe usage, prohibitions on usage and criteria for seeking professional consultation.

5. The method of claim 1, wherein one incentive offered to the consumer is a refund of purchase monies if the consumer is advised that the medicine may not be suitable for use by the consumer.

6. A method of qualifying and promoting appropriate over-the-counter administration of medicines by consumers, comprising the steps of:
a) offering unspecified incentives to consumers of the medicine as an inducement to the consumer to communicate with knowledgeable personnel regarding proper use of the medicine;
b) labeling such incentives as high value incentives; and
c) providing the consumer with the identification of interactive media through which the consumer may obtain information about the incentives.

7. The method of claim 56, wherein the interactive media facilitates a consumer's contact with a knowledgeable provider of information about the medicine.

8. A method of determining suitability of an over-the-counter (OTC) medicine for use by an individual consumer, the method comprising the steps of:
providing product information at a point of retail sale including multi-step guidance on qualifying criteria for use of said OTC medicine;
providing a telecommunications address for an interactive information service with packaging of said OTC medicine, said address being one of an Internet address and a telephone number;
carrying out interactive communications with said individual consumer at said interactive information service, wherein said consumer provides information to confirm compliance with said qualifying criteria;
confirming whether said individual consumer complies with said qualifying criteria using said information provided said individual consumer;
selecting one of a plurality of incentives based on said information provided by said individual consumer, said incentives including at least a partial rebate on a purchase price of said OTC medicine for said individual consumer in the case that said consumer does not comply with said qualifying criteria, and one or more incentives in the case that said consumer complies with said qualifying criteria; and
communicating to said individual consumer a result of said selecting, whereby appropriate over-the-counter administration of said OTC medicine by consumers is ensured and promoted.

9. The method as claimed in claim 8, wherein said telecommunications address is provided inside said packaging on a coupon.

10. The method as claimed in claim 8, wherein said telecommunications address uniquely identifies said OTC medicine.

11. The method as claimed in one of claims 8 to 10, wherein said interactive information service uses voice communications, and said consumer provides said information to confirm compliance with said qualifying criteria in response to a scripted series of questions.

12. The method as claimed in one of claims 8 to 11, wherein said one or more incentives in the case that said consumer complies with said qualifying criteria comprise one of: a free supply of said OTC medicine; and discounts on other products related to a condition being treated by said OTC medicine.

13. The method as claimed in one of claims 8 to 11, wherein said one or more incentives in the case that said consumer complies with said qualifying criteria comprise analytical tests related to the efficacy of said OTC medicine.

14. The method as claimed in claim 13, further comprising a step of transmitting a certificate redeemable for said analytical tests for said individual consumer.

15. The method as claimed in claims 8 to 14, wherein said at least one of said plurality of incentives is a high value incentive.

16. The method as claimed in claim 9, wherein said at least one of said plurality of incentives is a high value incentive and is identified as such on said coupon.
